(19) United States
(12) Patent Application Publication
(10) Pub. No.: US 2018/0022789 A1
(43) Pub. Date: Jan. 25, 2018

(54) GELATIN PARTICLES, METHOD FOR PRODUCING GELATIN PARTICLES, GELATIN-PARTICLECONTAINING CELLS, METHOD FOR PRODUCING GELATIN-PARTICLE-CONTAINING CELLS, AND CELLULAR STRUCTURE

(71) Applicants: Yasuhiko TABATA, Uji-shi (JP); Konica Minolta, Inc., Tokyo (JP)

(72) Inventors: Natsumi HIRAYAMA, Tokyo (JP); Chie INUI, Tokyo (JP); Akihiro MAEZAWA, Tokyo (JP); Yasuhiko TABATA, Uji-shi (JP)

(21) Appl. No.: 15/650,142

(22) Filed: Jul. 14, 2017

(30) Foreign Application Priority Data
Jul. 20, 2016 (JP) 2016-142668

(51) Int. Cl. C07K 14/78; A61K 9/48; A61K 9/00

(52) U.S. Cl. CPC C07K 14/78; A61K 9/0053; A61K 9/48; A61K 9/4808; A61K 9/0056; C07K 14/00

(57) ABSTRACT

Gelatin particles include: gelatin that serves as a main component; and an auxiliary component carried on the gelatin, the gelatin particles being configured such that where the particle size of the gelatin particles is X, the ratio A/B of the average concentration A (mass %) of the auxiliary component contained in a surface part having a thickness of 0.01X from the surface of the gelatin particles based on the total mass of the gelatin particles to the average concentration B (mass %) of the auxiliary component contained in an inner part of the particles deeper than the surface part based on the total mass of the gelatin particles is less than 0.25.

The entire disclosure of Japanese Patent Application No. 2016-142668 filed on Jul. 20, 2016 including description, claims, drawings, and abstract are incorporated herein by reference in its entirety.

### BACKGROUND OF THE INVENTION

### Field of the Invention

The present invention relates to gelatin particles, a method for producing gelatin particles, gelatin-particle-containing cells, a method for producing gelatin-particle-containing cells, and a cellular structure.

### Description of the Related Art

Gelatin is highly biocompatible and has the property of being degraded and easily absorbed in the body. Accordingly, a technology in which an additive and a drug (hereinafter sometimes simply referred to as “additive and the like”) are contained in gelatin in the form of particles and delivered in vivo, and these substances are released in vivo, has been developed.

For example, JP 2014-58465 A describes swollen gelatin particles composed of thermally crosslinked gelatin having a jelly strength of 80 to 120 g, wherein the average particle size of dry particles before swelling is 20 to 1,600 μm, and the average particle size of dry particles after swelling is 50 to 2,000 μm. According to JP 2014-58465 A, the swollen gelatin particles have excellent shape retention and are hard to break even if deformed due to external stress, and thus are suitable for administration into a blood vessel using a microcatheter or an injection needle.

In addition, JP 2008-510688 A describes gelatin particles composed essentially of an aqueous gelatin gel and having an average diameter of 350 nm or less with a narrow size distribution. According to JP 2008-510688 A, the gelatin nanoparticles are capable of selectively delivering and releasing the carried active substance, and thus are suitable for the targeted delivery of the active substance in vivo.

Further, Tomitaka, A. et al., “Preparation of biodegradable iron oxide nanoparticles with gelatin for magnetic resonance imaging”, Inflammation and Regeneration, 2014; Vol. 34, No. 1, pp. 45-55., describes iron oxide nanoparticles using gelatin and having an average particle size of 87 nm. According to Tomitaka, A. et al., “Preparation of biodegradable iron oxide nanoparticles with gelatin for magnetic resonance imaging”, Inflammation and Regeneration, 2014; Vol. 34, No. 1, pp. 45-55., the iron oxide nanoparticles were taken up into cells, and their presence was confirmable for 6 days after the uptake.

It is believed that the gelatin particles described in JP 2014-58465 A are suitable for so-called drug delivery system (DDS) applications, where the particles are administered inside blood vessels, organs, and the like to deliver and release the additive and the like.

Meanwhile, in recent years, there is an increasing demand for the technology to directly introduce an additive and the like into living cells. For example, when a contrast medium is introduced into cells, the cells' activity can be tested in a non-destructive manner. In addition, when cells having introduced thereinto a contrast medium are transplanted into a patient, whether the transplanted cells have colonized can be observed from outside in a minimally invasive manner without another incision of the transplantation site. Because gelatin is highly biocompatible, it is believed that gelatin particles are suitable also as a carrier for carrying an additive and the like to be introduced into living cells.

As methods for introducing gelatin particles carrying an additive and the like into cells, an electroporation method and a microinjection method are possible. However, according to these methods, the cell membrane is deformed to introduce the additive and the like into the cell membrane. Accordingly, the cell membrane may be partially destroyed, resulting in the loss of the cells' activity. In terms of minimizing the loss of the cells' activity, it is preferable that the additive and the like are taken up through the cells' own activity. For this purpose, it is desirable that gelatin particles carrying the additive and the like can also be easily taken up through the cells' own activity. However, according to the findings by the present inventors, the gelatin particles described in JP 2014-58465 A and JP 2008-510688 A are unlikely to be taken up into cells through the cells' own activity.

In addition, although the gelatin particles described in Tomitaka, A. et al., “Preparation of biodegradable iron oxide nanoparticles with gelatin for magnetic resonance imaging”, Inflammation and Regeneration, 2014; Vol. 34, No. 1, pp. 45-55., can be taken up into cells through the cells' own activity, according to the findings by the present inventors, their average particle size is small, and the amount of additive and the like that can be carried is limited. Therefore, there has been a demand for particles capable of carrying a large amount of additive and the like and sustained-releasing them inside living cells for a long period of time.

### SUMMARY OF THE INVENTION

An object of the present invention is to provide gelatin particles that carry an auxiliary component and are easily taken up through the cells' own activity, a method for producing such gelatin particles, cells containing such gelatin particles, a method for producing cells containing such gelatin particles, and a cellular structure containing cells containing such gelatin particles.

Problems of the prevent invention can be solved by the following means.
[1] To achieve the abovementioned object, according to an aspect, gelatin particles reflecting one aspect of the present invention comprises: gelatin that serves as a main component; and an auxiliary component carried on the gelatin, the gelatin particles being configured such that where the particle size of the gelatin particles is X, the ratio A/B of the average concentration A (mass %) of the auxiliary component contained in a surface part having a thickness of 0.01X from the surface of the gelatin particles based on the total mass of the gelatin particles to the average concentration B (mass %) of the auxiliary component contained in an inner part of the particles deeper than the surface part based on the total mass of the gelatin particles is less than 0.25.
[2] The gelatin particles of Item. 1, wherein the average concentration A of the auxiliary component contained in the surface part of the gelatin particles is preferably 5 mass % or less.
[3] The gelatin particles of Item. 1 or 2, wherein the average concentration B of the auxiliary component contained in the inner part of the gelatin particles is preferably 7 mass % or more and 30 mass % or less.
[4] The gelatin particles of any one of Items. 1 to 3, wherein the average particle size X of the gelatin particles is preferably 200 nm or more and 1000 nm or less.
[5] The gelatin particles of any one of Items. 1 to 4, wherein the auxiliary component is preferably a contrast medium.
[6] To achieve the abovementioned object, according to an aspect, a method for producing gelatin particles, reflecting one aspect of the present invention comprises:

in a solution containing gelatin that serves as a main component and a raw material of an auxiliary component, synthesizing an auxiliary component from the raw material, thereby giving a slurry containing the gelatin and the auxiliary component; and

adding a phase-separation inducing agent to the slurry, thereby forming the gelatin containing the auxiliary component into particles.
[7] The method for producing gelatin particles of Item. 6, wherein the slurry preferably has a gelatin concentration of 5 mg/ml or more and 100 mg/ml or less.
[8] The method for producing gelatin particles of Item. 6 or 7, wherein the phase-separation inducing agent is preferably added in an amount of 2 ml or more and 50 ml or less per ml of the slurry.
[9] The method for producing gelatin particles of any one of Items. 6 to 8, wherein the slurry preferably has an auxiliary component concentration of 1 mass % or more and 30 mass % or less.
[10] The method for producing gelatin particles of any one of Items. 6 to 9, wherein the auxiliary component is preferably a contrast medium.
[11] To achieve the abovementioned object, according to an aspect, a gelatin-particle-containing cell reflecting one aspect of the present invention comprises the gelatin particles of any one of Items. 1 to 5 inside the cell membrane.
[12] To achieve the abovementioned object, according to an aspect, a method for producing a gelatin-particle-containing cell, reflecting one aspect of the present invention comprises adding the gelatin particles of any one of Items. 1 to 5 and a cell to a liquid, and allowing the gelatin particles to be taken up inside the cell membrane of the cell through the cell's activity.
[13] To achieve the abovementioned object, according to an aspect, a cellular structure reflecting one aspect of the present invention contains the gelatin-particle-containing cell of Item. 11.
[14] The cellular structure of Item. 13, which is at least one selected from the group consisting of a cellular sheet in which a plurality of cells is aggregated in a sheet form, a spheroid in which a plurality of cells is aggregated in a spherical form, a cellular bead in which a cell population is wrapped with a membrane, and a cellular bead in which a cell is adhered to a surface of a bead.
[15] The cellular structure of Item. 13 or 14, wherein the cellular structure is formed from a mixture of the gelatin-particle-containing cell of Item. 11 and a polymer solution.

### DESCRIPTION OF THE PREFERRED EMBODIMENTS

Hereinafter, an embodiment of the present invention will be described in detail. However, the scope of the invention is not limited to the illustrated examples.

In order to solve the above problems, the present inventors have conducted extensive research about the conditions of gelatin particles that are easily taken up into cells by the cells themselves. As a result, the present inventors have found that the uptake through the cells' own activity is easy to achieve under the following conditions: in gelatin particles including gelatin that serves as a main component and an auxiliary component carried on the gelatin, where the particle size of the gelatin particles is X, the ratio A/B of the average concentration A of the auxiliary component contained in a surface part having a thickness of 0.01X from the surface of the gelatin particles to the average concentration B of the auxiliary component contained in an inner part of the particles deeper than the surface part is less than 0.25. The reasons therefor are believed to be as follows.

Gelatin is a biocompatible material. Accordingly, gelatin particles alone are unlikely to be recognized as a foreign substance by cells and are easily taken up into cells through endocytosis or like activity. However, when an auxiliary component, which is likely to be recognized as a foreign substance by cells and unlikely to be taken up, is exposed in a large amount on the gelatin particle surface, the uptake of such gelatin particles into cells through the cells' own activity is unlikely to be achieved. This tendency becomes more prominent with an increase in the average particle size of gelatin particles. In addition, in the case where the additive content in gelatin particles is set high in order to maintain the additive, such as a contrast medium, at a high concentration for a long period of time, generally, the additive is likely to be exposed on the gelatin particle surface. Here, when the additive concentration in the surface part is set sufficiently lower than the additive concentration in the inner part, the carried auxiliary component is mostly present in the inner part of the particles and is not or hardly present in the surface part. Thus, because there is no auxiliary component exposed on the particle surface, or even if present, the amount thereof is extremely small, such gelatin particles are unlikely to be recognized as a foreign substance by cells and are easily taken up into cells through the cells' own activity, presumably.

1. Gelatin Particles and Method for Producing the Same

This embodiment relates to gelatin particles and a method for producing gelatin particles.

1-1. Gelatin Particles

The gelatin particles according to this embodiment are gelatin particles including gelatin that serves as a main component and an auxiliary component carried on the gelatin. The gelatin particles are configured such that where the particle size of the gelatin particles is X, the ratio A/B of the average concentration A (mass %) of the auxiliary component contained in a surface part having a thickness of 0.01X from the surface of the gelatin particles based on the total mass of the gelatin particles to the average concentration B (mass %) of the auxiliary component contained in an inner part of the particles deeper than the surface part based on the total mass of the gelatin particles is less than 0.25. The gelatin particles having the above configuration are characterized in that they are easily taken up into cells as described below even when an auxiliary component that is difficult to take up through the cells' own activity is carried thereon. Therefore, the gelatin particles are also referred to as “easy-uptake gelatin particles” herein. The easy-uptake gelatin particles may be single particles or may also be in the form of a collection of a plurality of gelatin particles.

The main component of the easy-uptake gelatin particles is gelatin. Specifically, as analyzed with an amino acid analyzer, the particles contain 300 or more glycines out of 1,000 amino acid residues and also contain both alanine and proline. As long as the gelatin is capable of forming particles, any known gelatin may be used, including those obtained by modifying collagen derived from cow bone, cows kin, pigskin, pig tendon, fish scales, fish meat, and the like. Gelatin has been used for food and medical applications in the past, and its intake into the body hardly causes damage to the human body. In addition, gelatin is dispersed away in vivo and thus is advantageous in that removal from in vivo is not required. Incidentally, as long as the uptake of gelatin particles into cells is possible, the easy-uptake gelatin particles may contain components other than gelatin. Incidentally, with respect the amount of components other than gelatin, it is preferable that the amount is within a range where the damage caused by the intake into the body is negligible. In addition, it is preferable that the components other than gelatin are composed of substances that are not accumulated in vivo and are easily eliminated.

In terms of facilitating the formation of gelatin particles that satisfy the above conditions of average particle size and swelling degree, it is preferable that the weight average molecular weight of the gelatin forming the easy-uptake gelatin particles is 1,000 or more and 100,000 or less. The weight average molecular weight may be a value measured in accordance with the PAGI Method, 10^{th} ed., (2006).

The gelatin forming the easy-uptake gelatin particles may be crosslinked. The crosslinking may be crosslinking using a crosslinking agent or may also be self-crosslinking without using a crosslinking agent.

The crosslinking agent should be a compound having a plurality of functional groups that form a chemical bond with, for example, a hydroxyl group, a carboxyl group, an amino group, a thiol group, an imidazole group, or the like. Examples of such crosslinking agents include glutaraldehyde, water-soluble carbodiimides containing 1-ethyl-3-(3-dimethylaminopropyl)carbodiimide hydrochloride (EDC) and 1-cyclohexyl-3-(2-morpholinoethyl)carbodiimide-metho-p-toluene sulfonate (CMC), ethylene glycol diglycidyl ether, polyethylene glycol diglycidyl ether, compounds having two or more epoxy groups including polyglycerol polyglycidyl ether and glycerol polyglycidyl, and propylene oxide. Among them, in terms of further enhancing the reactivity, glutaraldehyde and EDC are preferable, and glutaraldehyde is more preferable.

The above self-crosslinking may be, for example, crosslinking by application of heat or by electron beam or UV irradiation.

The easy-uptake gelatin particles contain an auxiliary component carried on the gelatin. Examples of auxiliary components include contrast media used for applications such as the testing of bioactivity and the like, the measurement of substances in vivo, and the quantification of substances in vivo, semiconducting nanoparticles (quantum dots), and fluorescent carbon particles (carbon dots).

Examples of contrast media include magnetic substances used as contrast media for MRI. Examples of contrast media for MRI include contrast media containing gadolinium (Gd), iron (Fe₃O₄, y-Fe₂O₃, etc.), and the like.

The easy-uptake gelatin particles may further contain various drugs. Specific examples of drugs include proteins having pharmaceutical activity, plasmids, aptamers, antisense nucleic acids, ribozymes, and nucleic acids used for pharmaceutical applications, including tRNA, snRNA, siRNA, shRNA, ncRNA, and condensed DNA, as well as antigens used for pharmaceutical applications.

Examples of proteins having pharmaceutical activity include steroid, non-steroidal anti-inflammatory drugs (NSAID), vitamin A (retinoid), vitamin D3, vitamin D3 analogues, antibiotic substances, antiviral drugs, and antibacterial drugs.

B41673US01

Incidentally, the above drugs are more biocompatible as compared with the auxiliary components described above. Therefore, the influence of the content ratio thereof in the gelatin particles' surface part on the ease of uptake into cells is smaller. Therefore, the content ratio of such a drug and its distribution in gelatin particles should be determined according to the purposes, including the sustained-release properties in the cells, the release duration, and the like.

When the gelatin carries an auxiliary component, this means that the auxiliary component is immobilized on the gelatin particle surface or taken up into the gelatin particles. In this embodiment, where the average particle size of the easy-uptake gelatin particles is X, the ratio A/B of the average concentration A of the auxiliary component contained in the surface part having a thickness of 0.01X from the surface of the gelatin particles to the average concentration B of the auxiliary component contained in the inner part of the particles deeper than the surface part is less than 0.25. When the ratio A/B is less than 0.25, the carried auxiliary component is mostly present in the inner part of the particles, and the amount of auxiliary component present in the surface part of the particles is small. Because the amount of auxiliary component present in the surface part of the particles is small, it is believed that the auxiliary component is not exposed on the gelatin particle surface at all, or, even if exposed, the amount thereof is extremely small. It is believed that when gelatin particles are configured like this, even in the case of carrying a large amount of auxiliary component, the particles are unlikely to be recognized as a foreign substance by cells and are easily taken up into cells through endocytosis or like activity. From the above point of view, it is preferable that the ratio A/B of the average concentration A of the auxiliary component contained in the surface part of the gelatin particles to the average concentration B of the auxiliary component contained in the inner part is less than 0.1, more preferably less than 0.01.

It is preferable that the average concentration A of the auxiliary component contained in the surface part of the easy-uptake gelatin particles, that is, in a portion having a thickness of 0.01X (X is the average particle size) from the particle surface, is 5 mass % or less, more preferably 3 mass % or less, still more preferably 1.5 mass % or less, still more preferably 0.5 mass % or less, still more preferably 0.1 mass % or less, and still more preferably 0.01 mass % or less. When the average concentration A is 5 mass % or less, the amount of auxiliary component present in the surface part is relatively small, whereby the amount of auxiliary component exposed on the gelatin particle surface is reduced. As a result, the gelatin particles are unlikely to be recognized as a foreign substance by cells. This tendency becomes more prominent when the average concentration of the auxiliary component in the surface part of the gelatin particles is 1.5 mass % or less.

It is preferable that the average concentration B of the auxiliary component contained in the inner part of the easy-uptake gelatin particles, that is, in a portion deeper in the particles than the surface part (0.01X thick from the particle surface, wherein X is the average particle size) of the gelatin particles, is 1 mass % or more and 30 mass % or less, more preferably 7 mass % or more and 30 mass % or less, still more preferably 10 mass % or more and 30 mass % or less, and still more preferably that it is 10 mass % or more and 20 mass % or less. When the average concentration B is 10 mass % or more, it becomes possible to introduce a large amount of auxiliary component, which has been difficult to take up into cells through the cells' own activity. Further, it also becomes possible to sustained-release the auxiliary component inside the cells for a long period of time. In addition, when the average concentration B is 20 mass % or less, the amount of auxiliary component present in the surface part is not too large either, whereby the gelatin particles are unlikely to be recognized as a foreign substance by cells.

It is preferable that the average particle size of the easy-uptake gelatin particles is 200 nm or more and 1,000 nm or less. Although the easy-uptake gelatin particles carry an auxiliary component, because there is substantially no auxiliary component in the surface part, the particles are easily taken up into cells through the cells' own activity even when the average particle size is 1,000 nm. In order for a large number of gelatin particles to be taken up into cells within a shorter period of time, it is more preferable that the average particle size of the easy-uptake gelatin particles is 800 nm or less. Meanwhile, when the average particle size of the gelatin particles is 200 nm or more, an auxiliary component is easily carried within the particles, and the holding capacity for auxiliary components can be increased. From the above point of view, it is preferable that the average particle size of the gelatin particles is 300 nm or more.

It is preferable that the aspect ratio of the gelatin particles in dry state is 1.0 or more and 1.4 or less. When the aspect ratio is 1.4 or less, the gelatin particles are more likely to maintain the near-spherical shape before and after the swelling treatment, and, in a solution containing the gelatin particles and cells, the gelatin particles and cells are likely to come in contact at the contact surface with more uniform shape and size. Thus, there will be less difference in the ease of uptake among gelatin particles. Accordingly, it is believed that easy-uptake gelatin particles having the above aspect ratio make it easier to control the amount of gelatin particles taken up into cells and the amount of cells that take up the gelatin particles. The aspect ratio of the easy-uptake gelatin particles may be a value obtained by dividing the major axis of the gelatin particles by the minor axis of the gelatin particles.

Incidentally, as used herein, the average particle size, major axis, and minor axis of gelatin particles mean the particle size, major axis, and minor axis of dry gelatin particles after being allowed to stand in atmospheric air at 80° C. for 24 hours.

The minor axis and major axis of the easy-uptake gelatin particles may be values obtained by analyzing an image taken by a scanning electron microscope (SEM). The particle size of the easy-uptake gelatin particles may be a value obtained by averaging the major axis and minor axis of the gelatin particles. When the gelatin particles are in the form of a collection, the major axis, minor axis, particle size, and aspect ratio of the gelatin particles may be values obtained by the averaging the major axes, minor axes, particle sizes, and aspect ratios of a plurality of gelatin particles arbitrarily selected from the collection (e.g., 20 gelatin particles), respectively.

The average concentration A of the auxiliary component contained in the surface part of the easy-uptake gelatin particles and the average concentration B of the auxiliary component contained in the inner part can each be determined by XPS depth profile measurement. In the XPS depth profile measurement, X-ray photoelectron spectrometry (XPS) measurement and rare-gas ion sputtering, such as argon, are used together. As a result, while exposing the inner part of a sample, the surface composition can be successively analyzed. The distribution curve obtained by such measurement can be prepared, for example, by plotting the atomic ratio (unit: at %) of each element along the vertical axis and etching time (sputtering time) along the horizontal axis. Incidentally, in the element distribution curve where etching time is plotted along the horizontal axis like this, the etching time is almost correlated with the distance from the surface. Accordingly, it is possible that elemental analysis is performed from the surface of the easy-uptake gelatin particles to the center to determine the element distribution curve of the easy-uptake gelatin particles, and the amount of auxiliary component contained in the surface part is determined based on the element distribution from the measurement start point to the etching time corresponding to 0.01X (X is the average particle size), while the amount of auxiliary component contained in the inner part is determined based on the element distribution from the etching time corresponding to 0.01X to the etching time corresponding to the particle center.

At arbitrarily selected several points (e.g., 10 points), the amount of auxiliary component is measured by the above method. The average amount of auxiliary component (mass) contained in the surface part and that in the inner part are each determined, and then the concentration based on the total mass of the gelatin particles (i.e., the total mass of the gelatin and the auxiliary component) is determined. The obtained concentrations can be defined as the average concentration A and the average concentration B, respectively. By calculating the ratio of the average concentration A to the average concentration B, the ratio A/B can be obtained. In addition, when the gelatin particles are in the form of a collection, the average concentration A, average concentration B, and ratio A/B of the auxiliary component may be values obtained by averaging the average concentrations A, average concentrations B, and ratios A/B of a plurality of gelatin particles (e.g., 20 gelatin particles) arbitrarily selected from the collection, respectively.

1-2. Method for Producing Gelatin Particles

As a method for producing the easy-uptake gelatin particles, first, gelatin is formed into particles by [1] a method in which drops of a liquid containing dissolved gelatin (hereinafter sometimes simply referred to as “gelatin solution”) are discharged into the atmosphere in a heating tube or a drying chamber and dried (in-air dropping method), [2] a method in which drops of a gelatin solution are discharged into a hydrophobic solvent and dispersed (in-liquid dropping method), or [3] a method in which a gelatin solution is emulsified to disperse gelatin-containing microdrops (in-liquid dispersion method), for example. At the same time with, or alternatively after, the formation of gelatin particles by such a method, an auxiliary component is attached to and carried on the gelatin particles, thereby producing gelatin particles containing an auxiliary component.

Further, using the auxiliary-component-carrying gelatin particles produced by the above method as the core, a shell made of gelatin is provided therearound, whereby gelatin particles whose surface part formed of a shell contains substantially no auxiliary component can be produced. It is preferable that the thickness of the shell formed in this method is 1% or more of the average particle size X of the finally obtained gelatin particles (i.e., 0.01X or more).

In addition, according to the novel findings by the present inventors, the easy-uptake gelatin particles can also be produced by a method including: in a solution containing gelatin that serves as a main component and a raw material of an auxiliary component, synthesizing an auxiliary component from the raw material, thereby giving a slurry containing the gelatin and the auxiliary component; and adding a phase-separation inducing agent to the obtained slurry, thereby forming the gelatin containing the auxiliary component into particles. In this manner, it is possible to efficiently obtain gelatin particles configured such that the auxiliary component is uniformly dispersed in the gelatin particles, the ratio A/B of the average concentration A of the auxiliary component contained in the surface part having a thickness of 0.01X (X is the average particle size) from the surface of the gelatin particles to the average concentration B of the auxiliary component contained in the inner part of the particles deeper than the surface part is less than 0.25, and also the auxiliary component is monodispersed in the inner part of the particles. In addition, it is also possible to obtain easy-uptake gelatin particles that allow the above shell formation step to be omitted and can exhibit the desired performance.

For the synthesis of an auxiliary component in a solution containing gelatin to obtain a slurry containing gelatin and an auxiliary component, the solvent used to synthesize the auxiliary component and the reaction conditions vary depending on the auxiliary component. However, when the auxiliary component is synthesized in the presence of gelatin, a slurry in which the auxiliary component is not aggregated but monodispersed can be obtained. For example, in the case of producing gelatin particles carrying Fe₃O₄ as an auxiliary component, an aqueous solution containing FeCl₃.6H₂O and FeCl₂.4H₂O, which serve as raw materials of Fe₃O₄, as well as gelatin, is prepared, and an alkaline solution (e.g., solution of NaOH, NH₃, KOH, etc.) is added thereto to adjust the pH of the solution 7 or more, thereby synthesizing Fe₃O₄. As a result, a slurry having Fe₃O₄ uniformly dispersed in the gelatin solution is obtained.

In the step of adding a phase-separation inducing agent to the slurry obtained above and forming gelatin into particles, gelatin particles are formed by the coacervation of gelatin caused by the addition of the phase-separation inducing agent. The phase-separation inducing agent to be added to the slurry is not particularly limited as long as it is a component capable of forming gelatin into particles, and examples thereof include organic solvents, particularly alcohols, such as ethanol, 1-propanol, 2-propanol, and 1-butanol, and acetone.

In the method for producing gelatin particles described above including a step of synthesizing an auxiliary component in a solution containing gelatin, the average particle size of the obtained gelatin particles can be adjusted by the gelatin concentration in the slurry. With an increase in the gelatin concentration in the slurry, the average particle size of the obtained gelatin particles tends to increase. In addition, it is believed that the amount of phase-separation inducing agent added affects the homogeneity of the auxiliary component. In order to produce gelatin particles having an average particle size of 200 nm or more and 1,000 nm or less and having an auxiliary component uniformly dispersed therein, which are preferable as the easy-uptake gelatin particles, it is preferable that the gelatin concentration in the slurry is 5 mg/ml or more and 100 mg/ml or less, and the amount of phase-separation inducing agent added is 2 ml or more and 50 ml or less per ml of the slurry.

In addition, the amount of auxiliary component carried on the gelatin particles depends on the auxiliary component concentration in the slurry before forming gelatin into particles. When the content ratio of the auxiliary component is not too high, the auxiliary component concentration in the surface part of the gelatin particles obtained by this method is sufficiently smaller than the auxiliary component concentration in the inner part of the gelatin particles, whereby the uptake of gelatin particles into cells can be prevented from being inhibited. In order to produce gelatin particles configured such that the ratio A/B of the average concentration A of the auxiliary component contained in the surface part having a thickness of 0.01X (X is the average particle size) to the average concentration B of the auxiliary component contained in the inner part of the particles deeper than the surface part is less than 0.25, it is preferable that the concentration of the auxiliary component contained in the slurry is 1 mass % or more and 30 mass % or less.

The above method is particularly suitable in the case where the auxiliary component is a contrast medium.

In terms of further reducing the toxicity against living cells, it is preferable that the easy-uptake gelatin particles have low contents of an organic solvent and low-molecular-weight components derived from an organic solvent. For example, when the gelatin particles are dissolved in the eluent (0.05 M Na₂HPO₄+0.05 M KH₂PO₄, pH 6.8) and subjected to gel permeation chromatography (GPC) using Asahipak GS620 manufactured by Asahi Kasei Corporation (column length: 500 nm, column diameter: 7.6 mm, two columns) as the columns under the conditions of a column temperature of 50° C. and a flow rate of 1.0 cc/min using a UV absorption spectrophotometer (detection wavelength: 230 nm), it is preferable that the proportion of components having a molecular weight of 1,000 or less is 5% or less in the resulting molecular-weight distribution pattern.

2. Cells

This embodiment relates to cells containing easy-uptake gelatin particles inside the cell membrane, a method for producing such cells, and a cellular structure containing such cells.

2-1. Cells

Cells according to this embodiment are cells containing easy-uptake gelatin particles inside the cell membrane (hereinafter sometimes simply referred to as “gelatin-particle-containing cells”).

When gelatin particles are contained inside the cell membrane, this means that in an image of cells taken by TEM, gelatin particles are seen inside the cell membrane. With respect to the uptake of gelatin particles into cells, for example, in the case where the gelatin particles contain a contrast medium, the contrast medium is stained and microscopically observed, whereby whether the contrast-medium-containing gelatin particle have been taken up into the cells can be confirmed. In addition, in the case of gelatin particles not containing a contrast medium, the gelatin particles are previously fluorescently labeled, and whether the fluorescently labeled gelatin particles have been taken up into cells can be confirmed using a confocal microscope. The fluorescent labelling of gelatin particles can be performed using, as the substrate, FITC-gelatin prepared by mixing equal amounts of a solution labeled with fluorescein isothiocyanate (FITC) (e.g., a 10 mM acetic acid solution of FITC-collagen manufactured by Cosmo Bio Co., Ltd.), 0.4 M sodium chloride, 0.04% (W/V) sodium azide, and a 50 mM Tris-HCl buffer containing 10 mM calcium chloride (pH 7.5), followed by a heating treatment at 60° C. for 30 minutes, for example.

It is preferable that the easy-uptake gelatin particles contained in cells carry a contrast medium, particularly a contrast medium for MRI. Such cells are produced by the below-described method in which particles are taken up through the cells' own activity, and then the presence of the contrast medium in the cells is observed, whereby the cells' activity can be tested in a non-destructive manner.

As cells capable of containing gelatin particles inside the cell membrane, the following cells are usable: cells derived from biological samples or specimens extracted from various organs including the bone marrow, heart, lung, liver, kidney, pancreas, spleen, intestinal tract, small intestine, cardiac valve, skin, blood vessel, cornea, eyeball, dura mater, bone, trachea, and auditory ossicles; commercially available established cell lines; stem cells including skin stem cells, epidermal keratinocyte stem cells, retinal stem cells, retinal epithelial stem cells, cartilage stem cells, hair follicle stem cells, muscle stem cells, osteoprogenitor stem cells, preadipocyte stem cells, hematopoietic stem cells, nerve stem cells, hepatic stem cells, pancreatic stem cells, ectodermal stem cells, mesodermal stem cells, endodermal stem cells, mesenchymal stem cells, ES cells, and iPS cells; and known cells including cells differentiated from these stem cells.

Among these cells, in the case of cells to be transplanted into a patient in cell regenerative medicine, particularly stem cells or cells differentiated from stem cells, when such cells contain easy-uptake gelatin particles carrying a contrast medium, particularly a contrast medium for MRI, after transplantation into a patient, whether the gelatin-particle-containing cells have colonized the transplantation site can be observed by observing the contrast medium at the transplantation site without further surgery. Accordingly, it is believed that these cells, which contain gelatin particles carrying a contrast medium for MRI, can reduce the physical, mental, financial, and time burden on the patient who receives the regenerative medicine treatment, and enhance the quality of life (QOL) of the patient.

2-2. Method for Producing Cells

Gelatin-particle-containing cells can be produced by introducing easy-uptake gelatin particles into the above cells. Examples of methods for introducing gelatin particles into cells include a method in which gelatin particles and cells are added to a liquid, and the particles are taken up through the cells' own activity, such as endocytosis, and also a method in which they are introduced by external operation. Examples of methods in which the particles are taken up through the cells' own activity include a method in which gelatin particles and cells are stirred in a liquid and a method in which cells are cultured in a cell culture medium containing gelatin particles. Incidentally, the easy-uptake gelatin particles have a high uptake efficiency through cells themselves. Therefore, the operation of forming a complex with another component in order to promote the uptake into cells is not particularly necessary. In terms of minimizing the loss of the cells' activity, among the above methods, the method in which easy-uptake gelatin particles and cells are mixed in a liquid and cultured is preferable. Examples of methods in which particles are introduced by external operation include an electroporation method and a microinjection method. Among them, in terms of reducing the loss of the cells' activity during the introduction of gelatin particles, a method in which particles are introduced through the cells' own activity is preferable, and a method in which particles are taken up into cells without forming a complex is more preferable.

As the liquid to which gelatin particles and cells are added, a cell culture medium may be used. As the cell culture medium, it is possible to use a Hanks culture solution and a HEPES culture solution, for example. The cell culture medium may also be a known buffer or physiological saline. For example, it is possible to use a Hanks' balanced salt solution (HBSS), 4-(2-hydroxyethyl)-1-piperazineethanesulfonic acid (HEPES), and other known phosphate buffered saline (PBS).

In terms of enhancing the cells' activity and facilitating the uptake of gelatin particles into cells through the cells' own activity, it is preferable that the temperature of the cell culture medium during stirring is 15° C. or more and 50° C. or less, more preferably 35° C. or more and 45° C. or less.

When gelatin particles are introduced inside the cell membrane through the cells' own activity, for example, the cell culture medium containing the gelatin particles and the cells may be shaken so as to promote introduction.

Incidentally, it is believed that when gelatin particles are introduced through the cells' own activity, high-activity cells are more likely to take up gelatin particles, while low-activity cells are less likely to take up gelatin particles. Accordingly, by adding gelatin particles carrying a contrast medium and cells to a liquid, followed by shaking as necessary, and then observing whether there is any contrast medium inside the cells, the cells' activity can be tested in a non-destructive manner.

2-3. Cellular Structure

The gelatin-particle-containing cells can form a cellular structure in which a plurality of cells is gathered. The form of such a cellular structure is not particularly limited, and examples thereof include a cellular sheet as a two-dimensional culture, a spheroid (cell mass) as a three-dimensional culture, a cellular bead in which a cell population is wrapped with a membrane, and a cellular bead in which a cell is adhered to a surface of a bead. Components other than the cells contained in the cellular structure, such as the membranes and beads, are preferably made of a biocompatible material. Examples of the biocompatible material include polymer components such as laminin, proteoglycan, fibrin, matrigel, chitosan gel, polyethylene glycol, gelatin, and alginic acid.

The cellular structure having a three-dimensional structure can be formed from a mixture of gelatin-particle-containing cells and a polymer solution. Specifically, a polymer solution is prepared using one or more polymer components (e.g., laminin, proteoglycan, fibrin, matrigel, chitosan gel, polyethylene glycol, gelatin, and alginic acid), gelatin-particle-containing cells are embedded in the solution, and the resultant mixture is cultured. As a result, the cells turn into sheet-like or massive cultured cells, and such cultured cells are integrated to form a larger cell population. The population of the cultured cells thus formed can be used as a tissue-like cellular structure.

Cell types of the gelatin-particle-containing cells that form a cellular structure are not particularly limited, and examples thereof include skeletal muscle cells, smooth muscle cells, nerve cells, hepatocytes, cardiomyocytes, keratinocytes, and stem cells such as ES cells and iPS cells. In addition, the cellular structure is only required to contain at least one type of gelatin-particle-containing cells, and may contain two or more types of gelatin-particle-containing cells, or gelatin-particle-containing cells and other cells. For example, an organ-like three-dimensional cellular structure having a blood vessel can be obtained using gelatin-particle-containing cells for constructing tissues and cells for constructing blood vessels.

Such cellular structures can be transplanted to a patient as treatment in the field of cell regenerative medicine. In this case, the cellular structure contains cells containing easy-uptake gelatin particles carrying a contrast medium, in particular a contrast medium for MRI. Therefore, by observing the contrast medium at a transplantation site after the transplantation into the patient, it is possible to observe whether the cellular structures have colonized the transplantation site without further surgery. Therefore, it is believed that the cellular structure containing cells containing gelatin particles carrying a contrast medium for MRI can reduce the physical, mental, financial and time burdens on a patient undergoing regenerative medicine treatment, and enhance the quality of life (QOL) of the patient.

### EXAMPLES

Hereinafter, specific examples of the present invention will be described. Incidentally, the scope of the present invention is not construed as being limited to these examples.

1. Production of Gelatin Particles 1-1. Preparation of Raw Material Solution

Gelatin (G-2613P manufactured by Nitta Gelatin Inc.), FeCl₂.4H₂O (raw material of Fe²⁺), FeCl₃.6H₂O (raw material of Fe³⁺), and pure water were mixed according to the compositions shown in Table 1 below, thereby preparing raw material solutions. Next, 0.25 ml of a 28% aqueous NH₃ solution was added to each of the obtained solutions, and Fe₃O₄ was synthesized under the conditions of pH 9 and 40° C., thereby giving a gelatin slurry containing Fe₃O₄.

1-2. Production of Gelatin Particles

Acetone was added to each of the slurries produced above as a phase-separation inducing agent in the amount shown in Table 1 and mixed at 50° C. Particles precipitated in the slurry were recovered and washed with pure water, thereby giving Gelatin Particles 1 to 10.

Table 1 shows the concentrations and the amounts of gelatin, Fe²⁺, and Fe³⁺ used in the raw material solutions, as well as the amount of acetone added, used for the production of the Gelatin Particles 1 to 10.

TABLE 1
Production of Gelatin Particles 1 to 10

| | | | | | | | Phase-separation |
|---|---|---|---|---|---|---|---|
| | | | Fe²⁺ | | Fe³⁺ | | inducing agent |
| | Gelatin solution | | (FeCl₂ · 4H₂O) | | (FeCl₃ · 4H₂O) | | (acetone) |
| | Concentration | Amount used | Concentration | Amount used | Concentration | Amount used | Amount added |
| | (mg/ml) | (ml) | (mg/ml) | (ml) | (mg/ml) | (ml) | (ml) |
| Gelatin Particle 1 | 25 | 2.25 | 40 | 0.12 | 100 | 0.10 | 10 |
| Gelatin Particle 2 | 25 | 2.25 | 40 | 0.14 | 100 | 0.11 | 10 |
| Gelatin Particle 3 | 25 | 2.25 | 40 | 0.13 | 100 | 0.11 | 10 |
| Gelatin Particle 4 | 25 | 2.25 | 40 | 0.16 | 100 | 0.13 | 10 |
| Gelatin Particle 5 | 50 | 2.25 | 40 | 0.23 | 100 | 0.19 | 10 |
| Gelatin Particle 6 | 50 | 2.25 | 40 | 0.28 | 100 | 0.22 | 10 |
| Gelatin Particle 7 | 50 | 2.25 | 40 | 0.21 | 100 | 0.16 | 10 |
| Gelatin Particle 8 | 25 | 2.25 | 40 | 0.14 | 100 | 0.11 | 10 |
| Gelatin Particle 9 | 25 | 2.25 | 40 | 0.08 | 100 | 0.07 | 10 |
| Gelatin Particle 10 | 50 | 2.25 | 40 | 0.28 | 100 | 0.22 | 10 |

2. Measurement of Gelatin Particles

2-1. Average Particle Size

The Gelatin Particles 1 to 10 produced above were each imaged by a scanning electron microscope (SEM). The taken images were analyzed using an image-analysis particle size distribution software Mac-View manufactured by Mountech to measure the minor axes and major axes of arbitrarily selected 20 gelatin particles, and the averages were defined as the minor axis and major axis of each Gelatin Particle, respectively. The average of the minor axis and major axis of the Gelatin Particle was defined as the average particle size of each Gelatin Particle.

2-2. Average Concentration of Auxiliary Component

The Gelatin Particles 1 to 10 produced above were each subjected to X-ray photoelectron spectrometry (XPS), and the average concentration A of the auxiliary component contained in the surface part of the easy-uptake gelatin particles and the average concentration B of the auxiliary component contained in the inner part were each determined for arbitrarily selected 20 gelatin particles. The measurement conditions were as follows.

### (Conditions)

Etching ion species: Argon (Art)

Etching rate (in terms of SiO₂ thermally oxidized film): 0.05 nm/sec

Etching interval (in terms of SiO₂): 1 nm

X-ray photoelectron spectrometry device: Model Name: “VG Theta Probe”, manufactured by Thermo Fisher Scientific

Applied X-ray: Monocrystal spectral AlKa

X-ray spot diameter: 100 μm

Where the average particle size of gelatin particles is X, a portion having a thickness of 0.01X from the surface of the gelatin particles was defined as “surface part”, and a portion deeper than the surface part was defined as “inner part”. With respect to the surface part and inner part of each Gelatin Particle, the atomic concentration of Fe₃O₄, which is an auxiliary component, was measured at arbitrarily selected 10 points, and the averages thereof were defined as the average concentration A and average concentration B of the auxiliary component, respectively. Further, the ratio of the average concentration A to the average concentration B, that is, A/B, was determined.

3. Introduction into Cells and Evaluation

3-1. Introduction into Cells

50 ml of fetal bovine serum was added to 500 ml of a cell culture medium MEM Alpha basic (1X) manufactured by Life Technologies, and used as a cell culture medium. 1 mg of each of the Gelatin Particles 1 to 18 was added to 3 ml of the cell culture medium, and mouse osteoblast-derived cells (MC3T3E1) were added to a concentration of 6,000 cells/ml. The cell culture medium after cell addition was maintained at 40° C. for 24 hours, thereby preparing 18 evaluation samples.

3-2. Evaluation of Introduction by Cells

From each of the evaluation samples, some of the cell dispersion was isolated, and whether the gelatin taken up inside the cell membrane was confirmable was observed by the following procedures and judged according to the following criteria.

(Staining of Cells and Fe)

1 ml of 1% paraformaldehyde was added to the cultured cells to perform a cell immobilization treatment. Subsequently, 1 ml of a Fe-staining solution of the following composition was added to stain Fe. Further, 1 ml of a nuclear-staining solution adjusted to the following concentration was added to stain the cells.

(Composition of Fe-Staining Solution)

Equal volumes of the following two liquids were mixed to prepare the Fe-staining solution.

20 vol % HCL (5-fold dilution of concentrated hydrochloric acid) 10 mass % aqueous K₄ (Fe(CN₆)) solution (100 mg/(ml))

(Composition of Nuclear-Staining Solution)

5 parts by mass of ammonium sulfate and 0.1 parts by mass of Nuclear fast red were mixed with 100 parts by mass of distilled water to prepare the nuclear-staining solution.

(Counting of the Number of Cells That Have Taken Up Fe)

The stained cells were observed under an optical microscope to evaluate whether blue-stained Fe was contained in arbitrarily selected 20 cells.

⊙: Out of the 20 cells, 50% or more of the cells (10 or more cells) were confirmed to have taken up gelatin inside the cell membrane.

◯: Out of the 20 cells, 10% or more and less than 50% of the cells (2 or more and less than 10 cells) were confirmed to have taken up gelatin inside the cell membrane.

X: Out of the 20 cells, less than 10% of the cells ((less than 2 cells) were confirmed to have taken up gelatin inside the cell membrane.

XX: Out of the 20 cells, no cells were confirmed to have taken up gelatin.

With respect to the Gelatin Particles 1 to 10, Table 2 shows the average particle size, the average concentration A and average concentration B of the auxiliary component, the ratio A/B, and the uptake into cells after 24 hours.

[Table 2]

TABLE 2
Average Particle Size of Gelatin Particles 1 to 10,
Average Concentrations of Auxiliary Component, and Evaluation
Results of Uptake into Cells

| | Average | Concentration of auxiliary component | | | |
|---|---|---|---|---|---|
| | particle | Average | Average | | Uptake into |
| | size | concentration A | concentration B | | Cells |
| | nm | (mass %) | (mass %) | A/B | (after 24 hours) |
| Gelatin Particle 1 | 200 | 3.500 | 17.500 | 0.2 | ◯ |
| Gelatin Particle 2 | 200 | 2.091 | 20.909 | 0.1 | ◯ |
| Gelatin Particle 3 | 200 | 1.048 | 20.952 | 0.05 | ⊚ |
| Gelatin Particle 4 | 200 | 0.248 | 24.752 | 0.01 | ⊚ |
| Gelatin Particle 5 | 800 | 0.020 | 19.980 | 0.001 | ◯ |
| Gelatin Particle 6 | 800 | 0.011 | 22.989 | 0.0005 | ⊚ |
| Gelatin Particle 7 | 800 | 0.002 | 17.998 | 0.0001 | ⊚ |
| Gelatin Particle 8 | 200 | 4.600 | 18.400 | 0.25 | X |
| Gelatin Particle 9 | 200 | 7.333 | 14.667 | 0.5 | X |
| Gelatin Particle 10 | 800 | 4.600 | 18.400 | 0.25 | X X |

The Gelatin Particles 1 to 7, in which A/B (the auxiliary component concentration ratio between the surface part and inner part of the gelatin particles) was less than 0.25, were taken up into cells through the cells' own activity more easily as compared with the Gelatin Particles 8 to 10, in which the ratio A/B was 0.25 or more.

Comparing the Gelatin Particles 1 to 4 and the Gelatin Particles 5 to 7, when the average particle size was the same, the lower the ratio A/B was, the more easily the gelatin particles were taken up into cells through the cells' own activity. In addition, comparing the Gelatin Particle 4 having an average particle size of 200 nm and the Gelatin Particle 5 having an average particle size of 800 nm, the Gelatin Particle 4 having a higher ratio A/B was taken up into cells more easily. This is believed to be because smaller particles are more easily taken up into cells. However, like the Gelatin Particle 6 or 7 having an average particle size of 800 nm, by making the ratio A/B lower than 0.001, the uptake into cells was facilitated even in the case of relatively large gelatin particles. This is believed to be because of the following reason. The amount of auxiliary component present in the surface part of the gelatin particles is extremely small, and thus there is no auxiliary component exposed on the particle surface, or, even if present, the amount thereof is extremely small. As a result, such particles are unlikely to be recognized as a foreign substance by the cells, and thus easily taken up.

In addition, when the ratio A/B was 0.25 or more, the amount of gelatin uptake into cells decreased. Comparing the Gelatin Particles 8 and 10 having the same ratio A/B of 0.25, the uptake amount of the Gelatin Particle 10 having a larger average particle size was smaller.

The gelatin particles of the present invention can contain a contrast medium for MRI, for example, and be introduced into cells for transplantation used for regenerative medicine. Such cells are allowed to take up gelatin particles through the cells' own activity and imaged by MRI to observe whether the contrast medium is present inside the cells, whereby the cells' activity can be tested in a non-destructive manner. Therefore, it is believed that the gelatin particles of the present invention can reduce the disposal rate of cells used for regenerative medicine and enhance the utilization efficiency of the cells. In addition, when such cells or cellular structures containing the cells are transplanted, by imaging the transplantation site with MRI, whether the cells have colonized the transplantation site can be observed without further surgery. Accordingly, it is believed that the gelatin particles of the present invention can reduce the physical, mental, financial, and time burden on a patient and enhance the quality of life (QOL) of the patient.

Although the present invention has been described and illustrated in detail, it is clearly understood that the same is by way of illustrated and example only and is not to be taken byway of limitation, the scope of the present invention being interpreted by terms of the appended claims.

1. Gelatin particles comprising:
gelatin that serves as a main component; and
an auxiliary component carried on the gelatin,
the gelatin particles being configured such that where the particle size of the gelatin particles is X, the ratio A/B of the average concentration A (mass %) of the auxiliary component contained in a surface part having a thickness of 0.01X from the surface of the gelatin particles based on the total mass of the gelatin particles to the average concentration B (mass %) of the auxiliary component contained in an inner part of the particles deeper than the surface part based on the total mass of the gelatin particles is less than 0.25.

2. The gelatin particles according to claim 1, wherein the average concentration A of the auxiliary component contained in the surface part of the gelatin particles is 5 mass % or less.

3. The gelatin particles according to claim 1, wherein the average concentration B of the auxiliary component contained in the inner part of the gelatin particles is 7 mass % or more and 30 mass % or less.

4. The gelatin particles according to claim 1, wherein the average particle size X of the gelatin particles is 200 nm or more and 1000 nm or less.

5. The gelatin particles according to claim 1, wherein the auxiliary component is a contrast medium.

6. A method for producing gelatin particles, comprising:
in a solution containing gelatin that serves as a main component and a raw material of an auxiliary component, synthesizing an auxiliary component from the raw material, thereby giving a slurry containing the gelatin and the auxiliary component; and
adding a phase-separation inducing agent to the slurry, thereby forming the gelatin containing the auxiliary component into particles.

7. The method for producing gelatin particles according to claim 6, wherein the slurry has a gelatin concentration of 5 mg/ml or more and 100 mg/ml or less.

8. The method for producing gelatin particles according to claim 6, wherein the phase-separation inducing agent is added in an amount of 2 ml or more and 50 ml or less per ml of the slurry.

9. The method for producing gelatin particles according to claim 6, wherein the slurry has an auxiliary component concentration of 1 mass % or more and 30 mass % or less.

10. The method for producing gelatin particles according to claim 6, wherein the auxiliary component is a contrast medium.

11. A gelatin-particle-containing cell comprising the gelatin particles according to claim 1 inside the cell membrane.

12. A method for producing a gelatin-particle-containing cell, comprising adding the gelatin particles according to claim 1 and a cell to a liquid, and allowing the gelatin particles to be taken up inside the cell membrane of the cell through the cell's activity.

13. A cellular structure containing the gelatin-particle-containing cell according to claim 11.

14. The cellular structure according to claim 13, which is at least one selected from the group consisting of a cellular sheet in which a plurality of cells is aggregated in a sheet form, a spheroid in which a plurality of cells is aggregated in a spherical form, a cellular bead in which a cell population is wrapped with a membrane, and a cellular bead in which a cell is adhered to a surface of a bead.

15. The cellular structure according to claim 13, wherein the cellular structure is formed from a mixture of a polymer solution and the gelatin-particle-containing cell comprising gelatin particles inside the cell membrane, and
the gelatin particles comprise:
gelatin that serves as a main component; and
an auxiliary component carried on the gelatin,
the gelatin particles being configured such that where the particle size of the gelatin particles is X, the ratio A/B of the average concentration A (mass %) of the auxiliary component contained in a surface part having a thickness of 0.01X from the surface of the gelatin particles based on the total mass of the gelatin particles to the average concentration B (mass %) of the auxiliary component contained in an inner part of the particles deeper than the surface part based on the total mass of the gelatin particles is less than 0.25.
